(19) United States
(12) Patent Application Publication
(10) Pub. No.: US 2005/0221493 A1
(43) Pub. Date: Oct. 6, 2005

(54) Recombinant virus production for the manufacturing of vaccines

(75) Inventors: Ronald Vogels, Linschoten (NL); Menzo Jans Emco Havenga, Alphen a/d Rijn (NL); Sanne Melles, Den Haag (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/146,332

(22) Filed: Jun. 6, 2005

Related U.S. Application Data
(63) Continuation of application No. PCT/EP03/51034, filed on Dec. 17, 2003.

(30) Foreign Application Priority Data
Dec. 20, 2002 (EP) 02102681.8

(51) Int. Cl. C12N 7/00; C12N 15/86

(52) U.S. Cl. 435/456; 435/235.1

(57) ABSTRACT

The present invention relates to the production of recombinant viruses and/or recombinant viral proteins using cells that can grow in suspension and in serum-free conditions without the requirement of any animal- or human-derived components. In particular, the invention relates to the production of recombinant alphaviruses that are suitable for use in vaccines and in gene therapy applications. For example, Semliki Forest Virus particles carrying a heterologous gene of interest (e.g., an antigen) are produced on El-transformed non-tumorous human cells, preferably derived from primary retinoblasts, such as PER.C6™ cells.

### CROSS-REFERENCE TO RELATED APPLICATIONS

This application is a continuation of PCT International Patent Application No. PCT/EP2003/051034, filed on Dec. 17, 2003, designating the United States of America, and published in English, as PCT International Publication No. WO 2004/056979 A2 on Jul. 8, 2004, which application claims priority to European Patent Application Serial No. 02102861.8 filed Dec. 20, 2002, the entirety of each of which being incorporated herein by this reference.

### TECHNICAL FIELD

The invention relates generally to the fields of biotechnology and medicine; in particular, it relates to the development and manufacturing of vaccines and compositions for gene therapy. More in particular, the invention relates to the field of production of recombinant alphaviruses by using a human cell.

### BACKGROUND

Vaccination is the most important route of dealing with viral infections. Although a number of antiviral agents are available, these agents typically have limited efficacy. Administering antibodies against a virus may be a good way of dealing with viral infections once an individual is infected (passive immunization) and typically human or humanized antibodies do seem promising for dealing with a number of viral infections, but the most efficacious and safe way of dealing with virus infection is prophylaxis through “active immunization.” Active immunization is generally referred to as vaccination and vaccines comprise at least one antigenic determinant of typically a virus, preferably a number of different antigenic determinants of at least one virus or other pathogen, e.g., by incorporating in the vaccine at least one (viral) polypeptide or protein derived from the virus (subunit vaccines) or the other pathogen. Typically, these formats include adjuvants in order to enhance an immune response. This is also possible for vaccines based on whole virus (pathogen), for instance, in an inactivated form. Another possibility is the use of live attenuated forms of the pathogenic virus and a further possibility is the use of wild-type virus, for instance, in cases where adult individuals are not in danger from infection, but infants are and may be protected through maternal antibodies and the like. Other techniques that have been developed in the art are DNA vaccines or non-replicating recombinant viruses, while replication-competent viruses are feasible as well. Recombinant viruses can be based on the nucleic acid of the virus of interest. One could also envision a recombinant virus from a different source that is utilized as a carrier for the antigenic protein. One such platform is the use of recombinant adenoviruses, while another platform is based on poxviruses. These recombinant viruses generally give a good immune response in humans and they can harbor large heterologous nucleic acid inserts (generally the antigen). A third platform that is of high interest is based on alphaviruses. The alphavirus genus includes a number of viruses that are all members of the Togaviridae family. The genus includes Venezuelan Equine Encephalitis virus (VEE), Sindbis virus and Semliki Forest virus (SFV) as the three major species that have been studied extensively. Besides these three, several other alphaviruses have been identified: Ndumu virus, Buggy Creek virus, Highland J. virus, Fort Morgan virus, Babanki virus, Kyzylagach virus, Una virus, Aura virus, Whataroa virus, Bebaru virus, South African Arbovirus No. 86, Mayaro virus, Sagiyama virus, Getah virus, Ross River virus, Barmah Forest virus, Chikungunya virus, O'nyong-nyong virus, Western Equine Encephalitis virus (WEE), Middelburg virus, Everglades virus, Eastern Encephalitis virus (EEE), Mucambo virus and Pixuna virus. The alphaviruses are distributed worldwide and are generally found among humans, primates, rodents, birds, pigs and horses.

Alphaviruses have an unsegmented, 11 to 12 kb positive strand RNA genome, with a methylated cap-modified 5′-end and a 3′-end having a variable-length polyadenylation tract (for reviews, see Frolov et al. 1996 and Liljeström, 1994). The capsid of the virion is surrounded by a lipid envelope covered with a regular array of transmembranal protein spikes, each of which consists of a heterodimeric complex of two glycoproteins, E1 and E2. During viral replication in an infected host cell, the genomic (49S) RNA strand serves as a template for the synthesis of the complementary negative strand. This negative strand serves as a template for full-length genomic RNA and for an internally initiated positive-strand 26S sub-genomic RNA. The presence of these two strands in an infected host cell leads to massive amounts of proteins required for packaging new viral particles. In this process, the non-structural proteins Nsp-1 to -4 are translated from the 49S genomic RNA, while the structural proteins are translated from the sub-genomic 26S RNA as a polyprotein precursor (NH₂-C-p62-6K-E1-COOH) which is co-translationally cleaved in the capsid protein (C) by the capsid protein itself, and in the envelope proteins p62, 6K, and E1. In Semliki Forest Virus (SFV) and Sindbis virus, sequences at the 5′-end of the capsid gene function as a translational enhancer, providing a high expression level of the structural proteins. The C protein complexes with new viral genomes to form cytoplasmic nucleocapsid structures, while the spike proteins are translocated to the endoplasmic reticulum, where p62 and E1 dimerize and are routed to the cell surface where budding occurs. During transport to the cell surface, p62 is cleaved to its mature form E2 by a host cell protease. This cleavage is believed necessary for the infectivity of the particles.

Since the protein expression is so high, the protein levels may provoke early apoptosis of the host cell. The remnants of the apoptotic cells are subsequently cross-presented to T-cells by dendritic cells. Several features of the members of the alphavirus genus make them very useful for vaccination purposes: a) the wild-type virus is known to cause little disease in humans, b) a number of its species are very well studied and the genomic sequences are known, c) its RNA genome will not integrate in host cell genomes and d) the efficient expression of proteins encoded by the RNA results in efficient cross-presentation due to protein uptake and subsequent antigen presentation by dendritic cells in the vaccinated host. Furthermore, alphaviruses can be used as gene delivery vehicles in other settings, such as gene therapy in which it is needed to deliver a wild-type version of a gene to a cell lacking that wild-type gene, or in other possible gene therapeutic applications known in the art.

Generally, alphavirus field strains are isolated on primary avian embryo, for instance, chicken fibroblast cultures, while the isolated viruses are usually propagated on Baby Hamster Kidney cells (BHK-21) or on monkey cells (Vero). Also, recombinant alphaviruses can be produced on BHK-21 or on Vero cells. Examples of such production systems have been described in the art: U.S. Pat. No. 5,792,462 describes the use of helper cells for producing infectious but defective alphavirus particles; U.S. Pat. No. 5,739,026 describes recombinant RNA molecules that can be translated and replicated in animal host cells; while U.S. Pat. No. 6,156,558 and WO 01/81609 describe the use of combinations of immunizing components from alphaviruses to apply in vaccination methodology. It is obvious that using molecular biology techniques to produce and obtain recombinant alphavirus particles has been used extensively in the art. However, for large-scale recombinant alphavirus production, one needs a robust, high-throughput and safe platform on which one can produce high titers of recombinant (non-)replicating particles. Thus far, the art depended on animal-derived systems, namely Vero and BHK-21 cells. Both systems have been used extensively and yield proper amounts of alphavirus particles, but both have their disadvantages. BHK-21 is clearly not suited for safe vaccine production. It is an undefined cancerous hamster cell line derived from a kidney, its history and origin is vague, and vaccines produced on these cells are likely never to be regulatory-approved. Vero cells are monkey cells that have been applied in many different settings as well. The disadvantage of these cells is, amongst others, that they grow on micro-carriers, resulting in a laborious system for large-scale production, and that titers are relatively low. It is also known that alphaviruses are relatively toxic to Vero cells because they die relatively quickly after infection or transfection, resulting in low titers. Thus, there is a clear need in the art for a system that is safe, well defined, and clean, that is easy to handle, and that gives significant amounts of recombinant product for the use in vaccines and compositions applicable in gene therapy.

### SUMMARY OF THE INVENTION

In one embodiment, the invention provides methods and means for producing recombinant viruses, other than adenoviruses, that can be used for vaccination purposes as well as for gene therapeutic applications.

Preferably, the recombinant viruses that are produced with the methods and means of the invention are recombinant alphaviruses, such as Sindbis virus, Semliki Forest virus and Venezuelan Equine Encephalitis virus. Human cells are useful for producing such recombinant viruses. Preferred are methods in which the human cells are transformed with adenovirus nucleic acids such as the E1 region of adenovirus serotype 5.

The invention relates specifically to methods for producing a recombinant virus for use as a vector for heterologous nucleic acid delivery, comprising: a) providing a cell having at least a sequence encoding at least one gene product of the E1 region of an adenovirus, wherein the cell does not produce structural adenoviral proteins, with a nucleic acid encoding the recombinant virus; b) culturing the cell obtained in the previous step in a suitable medium; and c) allowing for expression of the recombinant virus in the medium and/or the cell.

The invention further relates to the use of a human cell having a nucleic acid sequence encoding at least one E1 protein of an adenovirus in its genome, which cell does not produce adenoviral structural proteins for producing a recombinant alphavirus or at least one recombinant alphaviral protein. It also relates to recombinant viruses obtainable by a method or a use according to the invention for use in a vaccine and in therapeutics for gene therapy. The invention further also relates to vaccine compositions comprising a recombinant virus according to the invention and to cells, such as human cells, having a sequence encoding at least one E1 gene product of an adenovirus in its genome, which human cell does not produce adenoviral structural proteins and which human cell comprises a nucleic acid encoding a recombinant virus.

### DESCRIPTION OF THE FIGURES

FIG. 1 shows the Green Fluorescent Protein activity in infected BHK-21 cells (A), Vero cells (B) and PER.C6™ cells (C) using BHK-21-produced recombinant EGFP-encoding Semliki Forest Viral particles, plus the rate of dead cells as detected upon infection in time.

FIG. 2 shows Semliki Forest Virus titers as calculated from viral batches obtained from RNA electroporation experiments using PER.C6™ cells and BHK-21 cells. Purified viruses were used in a subsequent titration using BHK-21 cells.

### DETAILED DESCRIPTION OF THE INVENTION

In one embodiment, the invention relates to methods and means for producing recombinant viruses other than adenoviruses using a human cell that has been transformed by the E1 region of an adenovirus, preferably the E1 region of adenovirus serotype 5. The invention provides a solution to at least part of the problems outlined above related to the field of recombinant virus production for vaccination purposes using mammalian cells. Methods of the present invention relate to the production of recombinant viruses based on at least two separate nucleic acids, each comprising genes required for the generation of a functional viral particle. Preferably, the methods of the present invention are used to produce recombinant alphaviruses that can be applied for prophylactic and/or therapeutic treatment of different kinds of infections, exemplified in a wide range of possible pathogenic entities, such as HPV, Marburg virus, Lassa virus, HIV, Ebola virus, RSV, malaria, influenza, coronaviruses, such as the SARS-causing virus, etc. Antigenic determinants from such pathogenic entities can be introduced into the genome of the alphavirus and thus subsequently delivered to an infected host cell. Apart for settings with heterologous nucleic acids encoding antigens, in one embodiment, the invention produces recombinant alphaviruses that replicate conditionally in cells wherein replication is required and that do not replicate in cells wherein the recombinant alphavirus should be silent.

It should be understood that the present invention relates to the production of “recombinant” viruses and, therefore, not to the production of viruses by infecting with a wild-type virus (and thus producing progeny from that infected virus). WO 01/38362 describes the use of particular cells that were originally designed for producing recombinant adenoviruses and for producing viruses by infecting the cells with wild-type (or re-assortant) viruses. Those produced viruses can be used for vaccination purposes thereafter. The present invention makes use of similar cells but, as described herein below, the cells are applied for producing “recombinant” viruses by introducing (preferably through transfection) nucleic acid(s) that encode the recombinant virus, wherein the recombinant adenovirus is not an adenovirus.

The term “alphavirus” has its normal meaning in the art and refers to the various species in the alphavirus genus, such as: Venezuelan Equine Encephalitis virus, Sindbis virus, Semliki Forest virus (SFV), Ndumu virus, Buggy Creek virus, Highland J. virus, Fort Morgan virus, Babanki virus, Kyzylagach virus, Una virus, Aura virus, Whataroa virus, Bebaru virus, South African Arbovirus No. 86, Mayaro virus, Sagiyama virus, Getah virus, Ross River virus, Barmah Forest virus, Chikungunya virus, O'nyong-nyong virus, Western Equine Encephalitis virus, Middelburg virus, Everglades virus, Eastern Encephalitis virus, Mucambo virus and Pixuna virus.

For SFV, a so-called “two-helper RNA system” has been described for producing recombinant viruses (Smerdou and Liljeström, 1999). This system is based on at least three separate DNA vectors or RNA transcribed therefrom that are introduced into a production host cell. The first vector is the replicon, which contains the replicase gene (non-structural proteins), the subgenomic promoter followed by the heterologous gene of interest (the gene encoding the antigen) and the 5′ and 3′ replication signals at both ends of the replicon. Besides this vector, two other (helper) vectors are being utilized, one of which contains a promoter followed by the capsid gene, while the second helper vector contains a promoter followed by the p62, 6K and E1 genes. For details of the different constructs and combinations and possible alterations within the vectors, see Smerdou and Liljeström (1999). It is to be understood that different combinations of structural proteins on the helper vectors are possible to come to the same results as described herein. Moreover, it is also possible to use a one-helper vector system or a system applying more than two helper vectors in the human cells as disclosed herein, as long as the occurrence of wild-type and/or replication-competent viruses is prevented. The invention is drawn to the use of an adenovirus E1-transformed human cell in combination with the introduction of nucleic acid-encoding viral proteins to have the human cell produce recombinant viral particles. Such viral particles can then subsequently be used for the generation of vaccines. The system of Smerdou and Liljeström (1999) is an example of a possible vector system and how such cells can be used for production. Clearly, viruses other than alphaviruses can be produced following similar lines of investigation using the present invention. Non-limiting examples of viruses, apart from alphaviruses that may be produced in a recombinant fashion by applying nucleic acid to the host cells and using the methods of the present invention are: Human Immunodeficiency virus (HIV), FIV, SIV, rubella virus, Marburg virus, Lassa virus, parainfluenza virus, measles virus, mumps virus, respiratory syncytial virus, human metapneumovirus, yellow fever virus, dengue virus, Hepatitis C Virus (HCV), Japanese encephalitis virus (JEV), tick-borne encephalitis virus, St. Louis encephalitis virus, West Nile virus, Herpes Simplex virus, cytomegalovirus, Epstein-Barr virus, Hanta virus, human Papillomavirus, rabies virus, human coronavirus, Ebola virus, smallpox virus and African swine fever virus. All viruses from the following (non-limited range of) virus families can potentially be produced by applying methods of the present invention: Retroviridae, Paramyxoviridae, Flaviviridae, Herpesviridae, Bunyaviridae; Hantaviridae, Papovaviridae, Rhabdoviridae, Coronaviridae, Arteriviridae, Filoviridae, Arenaviridae and Poxviridae.

The present invention relates to the production of recombinant viruses by using either a transient system or a system in which cells have been stably transfected with one or more helper vectors providing the complementing structural and/or non-structural components to build the required recombinant virus. The produced recombinant alphaviruses of the present invention may also be referred to as Viral Like Particles (VLPs) since they comprise a coat that is infectious but they do not comprise all elements required for full production of a new viral particle upon introduction into a host cell. Following the two-helper system outlined above for SFV, one should, for the transient set-up, envision a system in which the vectors in DNA form are transfected or electroporated or in any other way introduced into the host cell, followed by replication of the vectors and protein production resulting in viral particle formation. The introduction of the vectors can be either as DNA or as RNA transcribed from the DNA vectors, or both. If RNA is applied, it is preferably transcribed in vitro from the DNA vectors, using suitable promoters. If DNA is used to introduce into the host cells, the genes are preferably under the control of strong promoters resulting in high production levels of the encoded proteins. In another embodiment of the present invention, the invention provides a method wherein cells have been stably transfected with one or more helper vectors or wherein the replicase gene is under the control of an inducible promoter. In such a system, replication only occurs when the promoter is activated and the replicase gene and/or the heterologous gene are stably incorporated into the genome of the stable cell line. “Stably” as used herein means, in general, that the nucleic acid has been incorporated into the DNA of the cell line, for instance, in its chromosomes. The generated cell lines containing the helper vector(s) stably integrated in the genome can then subsequently be used for transfection, infection, electroporation or introduction in any other way of the replicon vector harboring the replicase gene and the heterologous gene of interest. Preferably, the genes are introduced by means of transfection. The replicase gene and the heterologous gene (together also referred to as the replicon) may be introduced into the stable cell lines of the present invention by means of infection. Infection of the replicon can be through different kinds of other recombinant viruses or viral-like particles. Examples of recombinant viruses that may be used for infecting stable lines of the present invention are adenoviruses that do not replicate in the infected cell. Because the cells of the present invention contain a nucleic acid with at least the part of the adenovirus E1 region that is able to transform and immortalize cells, the recombinant adenovirus that is used to deliver the replicon should be crippled in the E1 region as well as in another way to prevent replication of the adenovirus. In one embodiment, an adenovirus is used that comprises a deletion in the E2 region, for instance, in the E2A region. Such recombinant viruses can be produced on cells harboring a temperature-sensitive E2A gene (see WO 97/00326, WO 01/05945, WO 01/07571). Other deletions and mutations are also possible to prevent the adenovirus from replicating. This set-up enables one to reach high titers of the viral particle, the possibility of large-scale production with high consistency since the use of many variables (such as co-transfection of two, three or more different vectors) is excluded. Preferably, as mentioned, the stable cell lines of the present invention have been immortalized and transformed by the E1 region of adenovirus, or at least a part of E1 that is capable of immortalizing and transforming cells. More preferred are PER.C6™ cells or PER.C6™-like cells, or derivatives or descendants thereof. The stable cell lines of the present invention are preferably grown in suspension and under serum-free conditions, wherein the medium contains no animal- or human-derived components. The stable transfection of the helper vector(s) is preferably executed with selection markers that enable the selection of cells that stably incorporated the foreign DNA into its genome and that displays sufficiently high levels of the encoded protein(s). Preferred selection markers are the Neomycin resistance gene and the Hygromycin resistance gene, although other selection markers well known to persons skilled in the art may be applied.

A sequence is said to be “derived” as used herein if a nucleic acid can be obtained through direct cloning from wild-type sequences obtained from wild-type viruses, while they can, for instance, also be obtained through PCR by using different pieces of DNA as a template. This also means that such sequences may be in the wild-type form as well as in altered form. Another option for reaching the same result is through combining synthetic DNA. It is to be understood that “derived” does not exclusively mean a direct cloning of the wild-type DNA. A person skilled in the art will also be aware of the possibilities of molecular biology to obtain mutant forms of a certain piece of nucleic acid. The terms “functional part, derivative and/or analogue thereof” are to be understood as equivalents of the nucleic acid they are related to. A person skilled in the art will appreciate the fact that certain deletions, swaps, (point) mutations, additions, etcetera may still result in a nucleic acid that has a similar function as the original nucleic acid. It is, therefore, to be understood that such alterations that do not significantly alter the functionality of the nucleic acids are within the scope of the present invention.

“Packaging defective” as used herein means that the viral vectors do not package in non-complementing cells. The replicon is replicated to very high levels but cannot be packaged in cells that do not comprise the structural genes. In complementing cells, the functions required for packaging, and thus production of the viral vector, are provided by the complementing cell. The packaging-defective viruses of the present invention lack elements that are required for full packaging.

“Heterologous” as used herein in conjunction with nucleic acids means that the nucleic acid is not found in wild-type versions of the viral vectors in which the heterologous nucleic acid is cloned. For instance, in the case of alphaviruses, the heterologous nucleic acid that is cloned in the replication-defective alphavirus vector is not an alphaviral nucleic acid.

“Antigenic determinant” as used herein means any antigen derived from a pathogenic source that elicits an immune response in a host towards which the determinant is delivered (administered). These pathogenic sources can be bacteria, yeasts, parasites, viruses, etc. Non-limiting examples of pathogenic sources that can be selected to provide the antigenic determinant are Human Immunodeficiency Virus (HIV), SIV, an Ebola virus, a malaria-causing parasite (such as Plasmodium falciparum or Plasmodium yoelii), Japanese Encephalitis Virus (JEV), Herpes Simplex Virus (HSV), Human Papillomavirus (HPV), Marburg virus, Lassa virus, Hanta virus, a rotavirus or a metapneumovirus. Non-limiting examples of antigenic determinants that can be used to clone into the recombinant viral vectors of the present invention are, for instance, the gag, pol, env and/or nef proteins of HIV, the E6 and/or E7 proteins of HPV or the circumsporozoite (CS) protein of P. falciparum.

The introduction of the nucleic acid into the cell can be through different methods known in the art. Preferably, the nucleic acid is transfected. An even more preferred method is electroporation of DNA and/or RNA.

The present invention relates to methods for producing a recombinant virus for use as a vector for heterologous nucleic acid delivery, comprising: a) providing a cell having at least a sequence encoding at least one gene product of the E1 region of an adenovirus, wherein the cell does not produce structural adenoviral proteins, with a nucleic acid encoding the recombinant virus; b) culturing the cell obtained in the previous step in a suitable medium; and c) allowing for expression of the recombinant virus in the medium and/or the cell. In a preferred embodiment, the recombinant virus comprises a heterologous nucleic acid. More preferably, the nucleic acid that is provided to the cell comprises the heterologous nucleic acid. In an even more preferred embodiment, the heterologous nucleic acid encodes an antigen, wherein the antigen is preferably of a Human Immunodeficiency Virus (HIV), SIV, an Ebola virus, a malaria-causing parasite, Japanese Encephalitis Virus (JEV), Herpes Simplex Virus (HSV), Human Papillomavirus (HPV), a Lassa virus, a Marburg virus, a rotavirus or a metapneumovirus.

In one aspect of the invention, the cell that is provided with the purified nucleic acid in the methods of the present invention is derived from a non-tumorous human cell. Preferably, the cell is derived from a primary human embryonic retinoblast, wherein the sequence encoding at least a gene product of the E1 region is present in the genome of the cell. In a highly preferred embodiment, the cell is a PER.C6™ cell as represented by the cells deposited under ECACC No. 96022940 at the European Collection of Animal Cell Cultures (ECACC) at the Centre for Applied Microbiology and Research (CAMR, UK), or a derivative or descendant of such cells.

The purified nucleic acid that is provided to the cell might be in the form of RNA and/or DNA, wherein the nucleic acid is provided to the cells preferably by transfection, more preferably by electroporation. If stable cell lines are generated that comprise certain nucleic acids stably integrated into the genome, providing the nucleic acid to the cell might be performed by infection using another recombinant virus, wherein the other recombinant virus might be an adenovirus, and wherein the adenovirus is not complemented in the infected cell. To ensure that the adenovirus is not complemented in a cell line that comprises the E1 region of adenovirus, the backbone of the adenovirus genome should be crippled in such a way that replication, production of other adenoviral genes (such as genes coding for structural proteins) and/or packaging of possibly produced DNA is prevented. One preferred way of accomplishing this is by deletion of the functional parts of the E2 region, for instance, by deleting the functional part of the E2A gene. Other regions that may be (partly) deleted for purposes of space as well as for the prevention of replication, protein production and packaging, are the E3 and the E4 region. Therefore, in a preferred embodiment, the adenovirus used for providing the nucleic acid encoding at least a part of the recombinant virus to be produced (other than an adenovirus, and preferably further comprising a heterologous nucleic acid) comprises an adenoviral genome comprising a deletion in the E2 region, the E3 region and/or the E4 region.

In a preferred aspect of the invention, the methods of the invention are applied for producing a recombinant alphavirus, preferably selected from the group consisting of: Venezuelan Equine Encephalitis virus (VEE), Sindbis virus, Semliki Forest virus (SFV), Ndumu virus, Buggy Creek virus, Highland J. virus, Fort Morgan virus, Babanki virus, Kyzylagach virus, Una virus, Aura virus, Whataroa virus, Bebaru virus, South African Arbovirus No. 86, Mayaro virus, Sagiyama virus, Getah virus, Ross River virus, Barmah Forest virus, Chikungunya virus, O'nyong-nyong virus, Western Equine Encephalitis virus (WEE), Middelburg virus, Everglades virus, Eastern Encephalitis virus (EEE), Mucambo virus and Pixuna virus.

In another preferred aspect of the invention, methods are provided wherein the nucleic acid that is provided to the host cell comprises at least two separate nucleic acid molecules, preferably wherein at least one of the separate nucleic acid molecules is DNA and stably integrated into the genome of the cell. In an even more preferred embodiment, the integrated nucleic acid comprises at least two separate nucleic acid molecules and wherein the integrated nucleic acid molecule encodes at least one structural protein. Highly preferred is an embodiment wherein the integrated nucleic acid molecule encodes the capsid, p62, 6K or the E1 protein of an alphavirus, or any combination thereof.

In yet another embodiment, methods are provided, wherein at least one of the separate nucleic acid molecules is not integrated into the genome of the cell and wherein the non-integrated nucleic acid molecule encodes the replicase of an alphavirus. This replicon construct preferably comprises the heterologous nucleic acid.

The invention also relates to the use of a human cell having a sequence encoding at least one E1 protein of an adenovirus in its genome, which cell does not produce adenoviral structural proteins for producing a recombinant alphavirus or at least one recombinant alphaviral protein. Preferably, the human cell is derived from a primary retinoblast, such as PER.C6™ cells, represented by cells deposited under ECACC no. 96022940, or a derivative or descendant thereof.

The invention furthermore relates to recombinant virus obtainable by a method or a use according to the invention for use in a vaccine or in therapeutics that can be applied in gene therapy settings. Therefore, the invention also relates to vaccine compositions comprising a recombinant virus according to the invention and a pharmaceutically acceptable carrier, and optionally, further comprising an adjuvant. Pharmaceutically acceptable carriers as used in vaccines are well known in the art and widely used. Moreover, if an elevated immune response is required, an adjuvant can be used, which is a feature that is also known to persons skilled in the art.

In one embodiment, the invention also relates to human cells having a sequence encoding at least one E1 gene product of an adenovirus in its genome, which human cell does not produce adenoviral structural proteins and comprises a nucleic acid encoding a recombinant virus, wherein the recombinant virus is preferably an alphavirus selected from the group as disclosed herein and wherein the human cell is preferably a PER.C6™ cell or a derivative thereof. Preferably, the nucleic acid present in the human cells according to the invention is separated into at least two separate nucleic acid molecules, wherein preferably at least one of the two separate nucleic acid molecules is stably integrated into the genome of the human cell. In an even more preferred aspect, the integrated nucleic acid molecule is divided into at least two separate parts and wherein the integrated nucleic acid encodes at least one structural viral protein of the recombinant virus. Highly preferred is an aspect wherein the two separate parts each encodes at least one structural viral protein of the recombinant virus.

The invention is further explained with the aid of the following illustrative examples)

### EXAMPLES

### Example 1

### Semliki Forest Virus Production on PER.C6™ Cells

PER.C6™ cells (WO 97/00326, U.S. Pat. Nos. 5,994,128 and 6,033,908, deposited at the ECACC, no. 96022940) were originally generated by transfection of primary human embryonic retina cells with a plasmid containing the Adenovirus serotype 5 (Ad5) E1A- and E1B-coding sequences (Ad5 nucleotides 459-3510) under the control of the human phosphoglycerate kinase (PGK) promoter.

The following features make PER.C6™ or a derivative thereof particularly useful as a host for virus production: it is a fully characterized human cell line, it can be grown as suspension cultures in defined serum-free medium to very high densities, the applied medium is devoid of any human or animal serum proteins, its growth is compatible with roller bottles, shaker flasks, spinner flasks and bioreactors, with doubling times of less or equal to approximately 30 hours. Surprisingly, although the cells were generated for adenovirus production, PER.C6™ cells also sustain the growth of a variety of viruses, other than adenovirus. As has been described by the applicants in WO 01/38362, the E1-transformed cells also support the growth of a wide variety of viruses, such as numerous strains of influenza virus, rotavirus, measles virus and herpes simplex virus. Although the togaviridae, including VEE and EEE as alphaviruses, were mentioned as possible candidates to be produced on PER.C6™, this was not investigated at the time. Therefore, it was tested whether PER.C6™ could indeed sustain alphavirus production. More in particular, it was investigated whether PER.C6™ could sustain the growth of viruses other than adenovirus by introducing nucleic acid into the cells and thereby generating recombinant viruses.

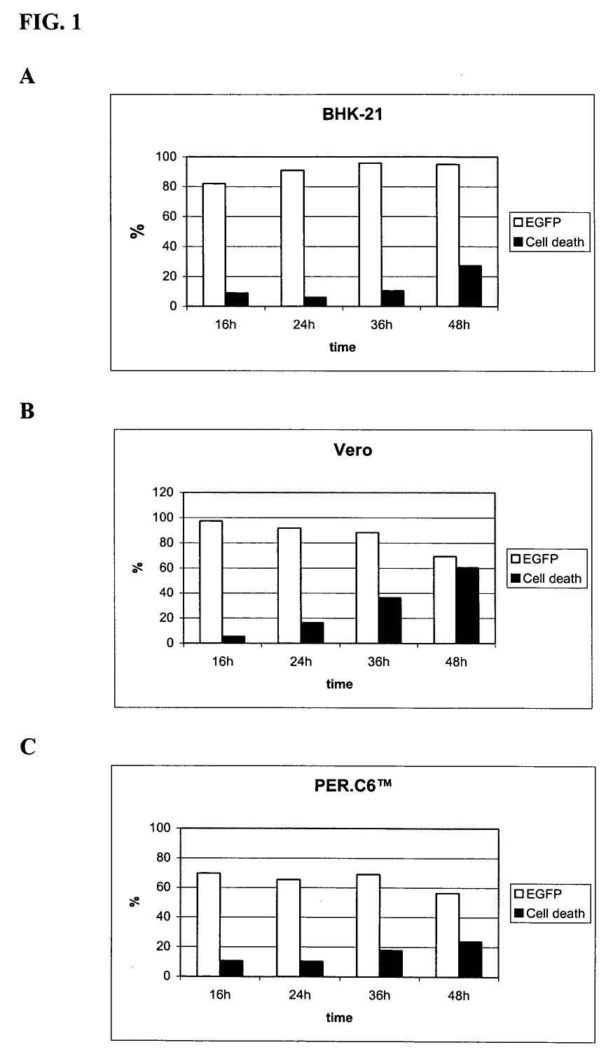

PER.C6™ cells were cultured from a master cell bank generally as previously described in WO 01/38362. First, the cells were tested for the ability of sustaining growth of Semliki Forest Virus particles. BHK-21 and Vero cells were taken as controls, since these cells are known in the art for the ability to produce recombinant alphaviruses. Cells were infected with a multiplicity of infection (moi) of 50 pfu/cell using a SFV particle harboring the Green Fluorescent Protein (GFP) construct, named SFV-EGFP (Liljeström and Garoff 1991). Cells were analyzed for GFP expression after 16, 24, 36 and 48 hours upon infection. Moreover, the cell death rate was determined at the same time points. FIG. 1A shows the GFP expression and death rate in infected BHK-21 cells. FIG. 1B shows the GFP expression and death rate in infected Vero cells. FIG. 1C shows the GFP expression and death rate in infected PER.C6™ cells. It should be noted that for PER.C6™, the same infection procedure was followed as was already optimized for BHK-21 cells. These results clearly show that E1-transformed human embryonic retinoblasts, such as PER.C6™, can support the replication of SFV replicons upon infection with SFV particles, while apparently the toxic effects of the alphaviral non-structural proteins (Nsp1-4) and replication events (in time after infection) as found in Vero cells (60% dead cells after 48 hours) are not as detrimental in PER.C6™ cells (approximately 20% dead cells after 48 hours). It remains to be determined what the production levels are, in time, compared between BHK-21, PER.C6™, and Vero cells to conclude what the titers will be on all cell lines. It remains to be determined what the speed of packaging is and at what stage cells are lysed due to the toxic effects of the nsp proteins and/or the replication and/or packaging events in the infected cells.

### Example 2

### Transient Electroporation of PER.C6™ Cells with RNA Encoding Semliki Forest Virus

PER.C6™ cells were cultured as described above. The cells were tested for the possibility to grow Semliki Forest Viruses upon introduction of nucleic acid encoding all essential components of an SFV particle. For this, cells were electroporated with RNA derived from the pSFV-Helper-1 construct and the pSFV-EGFP replicon comprising the replicase gene (Liljeström and Garoff, 1991; Smerdou and Liljeström, 1999). The electroporation protocol has been optimized for BHK-21 cells, but not for PER.C6™ cells. The protocol as described for BHK-21 will be further optimized for PER.C6™ cells by comparing differences in voltage, capacitance, time constant of electric pulse and the number of pulses given.

RNA was prepared as follows using an in vitro transcription kit and using methodology generally known in the art. Five μg of vector plasmid (based on pSFV-1, see Liljeström and Garoff, 1991) and 5 μg of the helper plasmid is linearized by digestion with the appropriate restriction enzyme (SpeI for pSFV-1 and the helper construct). The DNA is precipitated after phenol extraction by ethanol. Then the DNA is resuspended in water to a final concentration of 1.5 μg/μl. Five μl of this DNA solution is mixed with 5 μl 10×Sp6 buffer, 5 μl 50 mM DTT, 5 μl 10 mM m⁷G(5′)ppp(5′)G, 5 μl rNTP mix, 23 μl H₂O, 1.5 μl RNasin (50 units), 0.5 μl Sp6 RNA polymerase (30 units). This mixture was incubated at 37° C. for 60 to 90 minutes and produced RNA was checked on agarose gel. This protocol yields approximately 50 μg RNA per construct, which is the amount used for one electroporation. Aliquots are generally frozen at −80° C.

Electroporation was performed as follows. Cells were grown to a late log phase in their respective medium. Cells were washed once with PBS (without Mg²⁺ and Ca²⁺). For a 75 cm² bottle, 2 ml of trypsin was added and incubated at 37° C. until cells detached. Cells were then briefly pipetted such that single cells were obtained. The trypsin activity was stopped by the addition of 10 ml of normal medium. Cells were harvested by centrifugation and resuspended in PBS (without Mg²⁺ and Ca²⁺) to a concentration of 10⁷ cells per ml. 0.8 ml of this suspension was transferred to a tube containing the RNAs to be electroporated. Fifty (50) μl of in vitro transcribed RNA of each of the constructs were used. Cells and RNA were thoroughly mixed and transferred to a 0.4 cm electroporation cuvette. Pulse was set at 850 V and 25 μF and performed at room temperature. The time constant after the pulse was set at 0.4. Cells were subsequently diluted in their respective medium approximately 10 to 20-fold. The cuvette was rinsed to collect all cells. Cells were further maintained in a 75 cm² flask and incubated at 33° C. in a 5% CO₂ incubator for 48 hours to allow the cells to recover and release virus particles. It was found that for BHK-21 cells, it was best to culture the cells at this step at 33° C. instead of 37° C., because then a ten times higher titer could be obtained, probably because the onset of apoptosis is delayed. For PER.C6™ cells, this may be different but for now, not investigated. Thus, upon electroporation, cells were left for 48 hours after which the supernatant was harvested and SFV particles were purified and concentrated by ultra filtration. This was done as follows. The medium was collected and freed from cells and debris by centrifugation at 40,000 g for 30 minutes at 4° C. Supernatant was aliquoted and frozen on dry ice. Storage was done at −80° C.

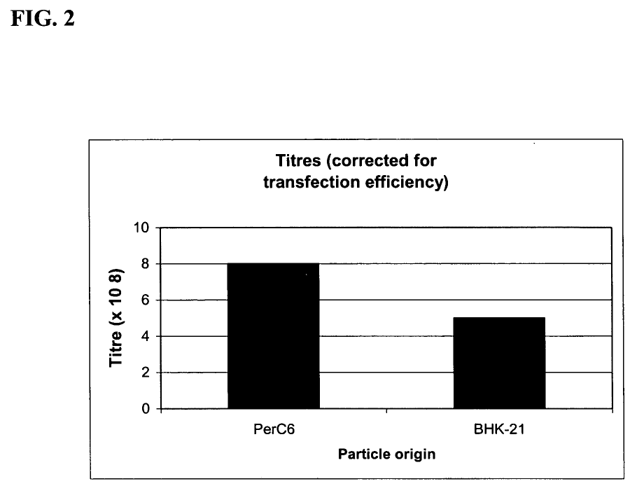

Purification and concentration of SFV particles was performed as follows. The viral supernatant was transferred to ultracentrifuge tubes (35 ml Beckman 25×89 mm tubes are suitable). Five ml 20% sucrose was added onto the bottom of the tube. The tube was further filled with medium. Spinning was performed at 140,000 g for 90 minutes at 4° C. Then, the tube was tilted and medium and sucrose fraction is removed. The virus pellet was resuspended in 0.25 to 0.5 ml TNE buffer. The concentrated virus stock was concentrated through a 0.22 μm filter, using a small syringe. Next, these purified particles were diluted sequentially in 10⁻¹, 10⁻², 10⁻³ and 10⁻⁴ (ten-fold) dilutions and used for BHK-21 infections. FIG. 2 shows the results of these subsequent BHK-21 infection experiments, using FACS analysis to determine GFP-positive cells (18 hours after infection) with the four ten-fold diluted samples derived from either PER.C6™ or BHK-21 cells. An average was determined between the four samples calculating from the number of particles per electroporated cell. The numbers were thus corrected for electroporation efficiency, resulting in a titer of approximately 8×10⁸ pfu for PER.C6™ cells and 5×10⁸ pfu for BHK-21 cells. Calculation was as follows: uncorrected particle titers obtained for PER.C6™ were 4×10⁷ and for BHK-21 5×10⁸. Calculation of the PER.C6™ titer was done by correcting with the transfection efficiency seen when electroporating PER.C6™ with SFV RNA, which turned out to be only 5% under these non-optimized conditions. Therefore, 4×10⁷ times 20 gives 8×10⁸ and for BHK (95% transfection efficiency) gives 5×10⁸ divided by 0.95 gives 5.2×10⁸. Apparently, many PER.C6™ cells died during the electroporation procedure, while approximately only 5 to 20% of the surviving cells were found positive for receiving SFV RNA. Clearly, the procedures to introduce RNA and/or DNA encoding SFV structural and non-structural components can still be optimized for PER.C6™ cells. Nevertheless, the results shown here indicate that PER.C6™ cells are able to sustain the growth of biologically active recombinant Semliki Forest Viruses, thereby providing a new and potent tool for producing large-scale batches of alphaviruses that can be used for producing safe vaccines directed against any pathogenic entity of interest.

### REFERENCES

Frolov I., T. A. Hoffman, B. M. Pragai, S. A. Dryga, H. V. Huang, S. Schlesinger and C. M. Rice (1996) Alphavirus-based expression vectors: strategies and applications. Proc. Natl. Acad. Sci. U.S.A. 93:11371-11377.
Liljeström P. (1994) Alphavirus expression systems. Curr. Opin. Biotechnol. 5:495-500.
Liljeström P. and H. Garoff (1991) A new generation of animal cell expression vectors based on the Semliki Forest virus replicon. Biotechnology (NY) 9:1356-1361.
   Smerdou C. and P. Liljeström (1999) Two-helper RNA system for production of recombinant Semliki Forest Virus particles. J. Virol. 73:1092-1098.

1. A method for producing a recombinant alphavirus for use as a vector for heterologous nucleic acid delivery, said method comprising:
a) providing a cell having at least a sequence encoding at least one gene product of the E1 region of an adenovirus, wherein said cell does not produce structural adenoviral proteins, with a nucleic acid sequence encoding said recombinant alphavirus;
b) culturing the cell in a suitable medium; and
c) allowing for expression of said recombinant alphavirus in said medium and/or said cell.

2. The method according to claim 1, wherein said recombinant alphavirus comprises a heterologous nucleic acid sequence.

3. The method according to claim 2, wherein said heterologous nucleic acid sequence encodes an antigen.

4. The method according to claim 3, wherein said antigen is of a virus selected from the group consisting of Human Immunodeficiency Virus (HIV), SIV, an Ebola virus, a malaria causing parasite, Japanese Encephalitis Virus (JEV), Herpes Simplex Virus (HSV), Human Papilloma virus (HPV), a Lassa virus, a Marburg virus, a rotavirus, a (SARS-causing) coronavirus, and a metapneumovirus.

5. The method according to claim 1, wherein the cell in step a) is derived from a non-timorous human cell.

6. The method according to claim 1, wherein the cell in step a) is derived from a primary human embryonic retinoblast.

7. The method according to claim 1, wherein said sequence encoding at least a gene product of the E1 region is present in the genome of said cell.

8. The method according to claim 1, wherein the cell in step a) is a PER.C6™ cell as represented by cells as deposited under ECACC no. 96022940, or a derivative thereof.

9. The method according to claim 1, wherein said nucleic acid sequence encoding said recombinant alphavirus is RNA.

10. The method according to claim 1, wherein said nucleic acid sequence encoding said recombinant alphavirus is DNA.

11. The method according to claim 1, wherein said nucleic acid sequence encoding said recombinant alphavirus is provided by transfection.

12. The method according to claim 1, wherein said nucleic acid sequence encoding said recombinant alphavirus is provided by electroporation.

13. The method according to claim 1, wherein said alphavirus is selected from the group consisting of Venezuelan Equine Encephalitis virus (VEE), Sindbis virus, Semliki Forest virus (SFV), Ndumu virus, Buggy Creek virus, Highland J. virus, Fort Morgan virus, Babanki virus, Kyzylagach virus, Una virus, Aura virus, Whataroa virus, Bebaru virus, South African Arbovirus No. 86, Mayaro virus, Sagiyama virus, Getah virus, Ross River virus, Barmah Forest virus, Chikungunya virus, O'nyong-nyong virus, Western Equine Encephalitis virus (WEE), Middelburg virus, Everglades virus, Eastern Encephalitis virus (EEE), Mucambo virus, and Pixuna virus

14. The method according to claim 13, wherein said alphavirus is a Semliki Forest Virus, a Sindbis Virus, or a Venezuelan Equine Encephalitis virus.

15. The method according to claim 1, wherein said nucleic acid sequence encoding said recombinant alphavirus comprises at least two separate nucleic acid molecules.

16. The method according to claim 15, wherein at least one of said at least two separate nucleic acid molecules is DNA and stably integrated into the genome of said cell.

17. The method according to claim 16, wherein said integrated nucleic acid molecule comprises at least two separate nucleic acid molecules.

18. The method according to claim 16, wherein said integrated nucleic acid molecule encodes at least one structural protein.

19. The method according to claim 18, wherein said integrated nucleic acid molecule encodes the capsid, p62, 6K, the E1 protein of an alphavirus, or any combination thereof.

20. The method according to claim 15, wherein at least one of said separate nucleic acid molecules is not integrated into the cell's genome.

21. The method according to claim 20, wherein said non-integrated nucleic acid molecule encodes the replicase of an alphavirus.

22. The method according to claim 20, wherein said non-integrated nucleic acid molecule comprises said heterologous nucleic acid sequence.

23. A method of producing of a recombinant alphavirus or at least one recombinant alphaviral protein, said method comprising:
using a human cell having a sequence encoding at least one E1 protein of an adenovirus in its genome, which cell does not produce adenoviral structural proteins for the production of a recombinant alphavirus or at least one recombinant alphaviral protein.

24. The method according to claim 23, wherein said human cell is derived from a primary retinoblast.

25. The method according to claim 23, wherein said human cell is a PER.C6™ cell as represented by cells as deposited under ECACC no. 96022940, or a derivative thereof.

26. A vaccine comprising:
a recombinant alphavirus obtainable by the method according to claim 1 presented in a form suitable for administration to a mammal.

27. The vaccine of claim 26 further comprising:
a pharmaceutically acceptable carrier, and
an adjuvant.

28. A human cell having a sequence encoding at least one E1 gene product of an adenovirus in the human cell's genome, which human cell does not produce adenoviral structural proteins but which human cell does comprise a nucleic acid sequence encoding a recombinant alphavirus.

29. The human cell of claim 28, wherein said nucleic acid sequence encoding a recombinant alphavirus is separated into at least two separate nucleic acid molecules.

30. The human cell of claim 29, wherein at least one of said two separate nucleic acid molecules is stably integrated into the genome of said human cell.

31. The human cell of claim 30, wherein said integrated nucleic acid molecule is divided into at least two separate parts.

32. The human cell of claim 30, wherein said integrated nucleic acid encodes at least one structural viral protein of said recombinant alphavirus.

33. The human cell of claim 31, wherein said two separate parts each encodes at least one structural viral protein of said recombinant alphavirus.

34. The human cell of claim 28, wherein said alphavirus is selected from the group consisting of Venezuelan Equine Encephalitis virus (VEE), Sindbis virus, Semliki Forest virus (SFV), Ndumu virus, Buggy Creek virus, Highland J. virus, Fort Morgan virus, Babanki virus, Kyzylagach virus, Una virus, Aura virus, Whataroa virus, Bebaru virus, South African Arbovirus No. 86, Mayaro virus, Sagiyama virus, Getah virus, Ross River virus, Barmah Forest virus, Chikungunya virus, O'nyong-nyong virus, Western Equine Encephalitis virus (WEE), Middelburg virus, Everglades virus, Eastern Encephalitis virus (EEE), Mucambo virus, and Pixuna virus.

35. The human cell of claim 28, wherein said human cell is a PER.C6™ cell as represented by cells as deposited under ECACC no. 96022940, or a derivative thereof.
